(19) United States
(12) Patent Application Publication
(10) Pub. No.: US 2024/0035991 A1
(43) Pub. Date: Feb. 1, 2024

(54) XRF QUANTITATIVE ANALYSIS METHOD OF HEAVY METAL ELEMENTS BASED ON LLE-SVR

(71) Applicant: Yangtze Delta Region Institute (Huzhou), University of Electronic Science and Technology of China, Huzhou City (CN)

(72) Inventors: Fusheng LI, Huzhou City (CN); Wanqi YANG, Huzhou City (CN); Yanchun ZHAO, Huzhou City (CN); Rongjiang TANG, Huzhou City (CN); Lu GAN, Huzhou City (CN)

(21) Appl. No.: 18/186,035

(22) Filed: Mar. 17, 2023

(30) Foreign Application Priority Data
Jul. 26, 2022 (CN) 202210881518.5

(51) Int. Cl. G01N 23/223; G06N 20/10; G01N 33/24

(52) U.S. Cl. CPC G01N 23/223; G06N 20/10; G01N 33/24; G01N 2223/616

(57) ABSTRACT

Methods of XRF quantitative analysis of heavy metal elements based on LLE-SVR, such as may include: establishing a relationship between peak information and element content by using Local Linear Embedding Dimensionality For Reduction and Support Vector Regression Predictive Algorithms based on machine learning, to quantitatively analyze the content information of elements contained in substances.

### CROSS-REFERENCE TO RELATED APPLICATIONS

Benefit and priority under 35 U.S.C. § 119(a) is hereby claimed to Chinese Patent Application No. CN 202210881518.5 filed on Jul. 26, 2022, which is hereby incorporated by reference herein in the entirety.

### TECHNICAL FIELD

The present disclosure belongs to a technical field of element detection, and in particular, relates to a method for XRF quantitative analysis of heavy metal elements based on LLE-SVR.

### BACKGROUND ART

At present, toxic elements such as, cadmium, mercury, lead, arsenic, zinc, chromium, nickel, copper commonly exist in contaminated soil, which are difficult to be removed by soil microorganisms. These contaminants are easily transferred to agricultural products such as rice, corns, thereby entering to human food supply chain and resulting in food poisoning or even carcinogenic risk. The heavy metal pollution has seriously affected human life, and its characteristics of being difficult to degrade, easy to accumulate and high toxicity have a significant influence on the growth, yield and quality of crops. Therefore, the first step of treatment and remediation is to determine the content of various heavy metals in contaminated soil.

ED-XRF, one of the most commonly used instruments for elemental analysis, allows for nondestructive analysis and study of the composition of substances by using X-ray to excite the substances to produce X-ray fluorescence without destroying the sample. It has the advantages of fast analysis, high precision and good reproducibility, and has important application value in alloy detection, environmental protection and safety, explosives detection, medical testing, mineral analysis and other fields. Considering that heavy metal elements in soil are microelements and have a wide variety of species, it is easy to overlapping element peak counts. It is necessary to design a new algorithm to accurately determine the heavy metal content due to the existing conventional methods based on statistical principles and lack of data verification.

Through the above analysis, the problems and defects existing in the prior art are: inaccurate measurement results from the existing instrument measurement method, peak overlapping interference between elements, great error, and inability to accurately predict the element content of analytes to be measured.

### SUMMARY

For the existing problem in the prior art, the present disclosure provides methods for XRF quantitative analysis of heavy metal elements based on LLE-SVR.

The present disclosure is realized by methods for XRF quantitative analysis of heavy metal elements based on LLE-SVR, comprising: e.g., establishing the relationship between peak information and element content by using Local Linear Embedding For Dimensionality Reduction and Support Vector Regression Predictive Algorithms based on machine learning, to quantitatively analyze the content information of elements contained in substances.

Further, the method for XRF quantitative analysis of heavy metal elements based on LLE-SVR may comprise the following procedures and/or steps of:
a first step of obtaining a soil sample set and constructing an element set based on the soil sample set, and using ED-XRF Fluorescence Spectrometer to identify peak information and content information of the elements in the sample to be measured of the soil sample set corresponding to the element set, to obtain the measured component value and the content value of each element;
a second step of constructing an LLE-SVR model, and determining an input matrix and an output matrix of the LLE-SVR model, and normalizing the input matrix and the output matrix of the LLE-SVR model to obtain the normalized matrixes of the input matrix and the output matrix;
a third step of searching for a neighbor point and calculating a weight value of the neighbor point based on the normalized matrixes of the input matrix and the output matrix, and performing LLE for dimensionality reduction and calculating a component matrix after LLE for dimensionality reduction;
a fourth step of performing nonlinear function mapping and constructing a classification hyperplane, and introducing a penalty factor and a slack variable for constraint, and training the LLE-SVR model via parameter optimization to quantitatively predict the element content; and
a fifth step of denormalizing the normalized output matrix of the target element content to obtain the denormalized output matrix, and calculating the coefficient of determination to evaluate the predicting effect of the LLE-SVR model.

Further, the first step of obtaining the soil sample set and constructing the element set based on the soil sample set may comprise:
obtaining a standard sample set, and identifying all elements of all samples to be measured in the standard sample set by using ED-XRF Fluorescence Spectrometer, to obtain an element set A containing all elements of all samples to be measured in the standard sample set;
   wherein said all elements are element Nos. 12-92 in periodic table;
   the input matrix of the LLE-SVR model in the second step is a matrix composed of the measured component values of all target elements of the element set A and their corresponding interfering elements;
   the output matrix of the LLE-SVR model is a matrix composed of the measured content values of the target elements;
   the target elements are the elements to be quantitatively analyzed;
   the interfering elements are the elements that interfere with the target elements;
   the matrix of the measured component values is a matrix composed of the component values of m element(s) contained in each of n sample(s) to be measured;
   the first column of the matrix of the measured component values is the measured component values of a single target element, and the remaining m-1 column(s) of the matrix of the measured component values is composed of the measured component values of other target elements and the interfering elements corresponding to all target elements; and
the matrix of the measured content values is a matrix composed of the concentration values of the single target element contained in each of n samples to be measured.

Further, the second step of normalizing the input matrix and the output matrix of the LLE-SVR model may comprise:
normalizing the input matrix and the output matrix to obtain the normalized matrixes:

$${x}_{ij}^{′} = \frac{{x}_{ij} - {x}_{min}}{{x}_{max} - {x}_{min}} ;$$

$${\overline{X}}_{nm} = [ \begin{matrix}{x}_{1}^{{′}^{T}} \\ {x}_{2}^{{′}^{T}} \\ ⋮ \\ {x}_{n}^{{′}^{T}}\end{matrix} ] = {[ \begin{matrix}{x}_{11}^{′} & {x}_{12}^{′} & ⋯ & {x}_{1 m}^{′} \\ {x}_{21}^{′} & {x}_{22}^{′} & ⋯ & {x}_{2 m}^{′} \\ ⋮ & ⋮ & & ⋮ \\ {x}_{n 1}^{′} & {x}_{n 2}^{′} & ⋯ & {x}_{nm}^{′}\end{matrix} ]}_{n \times m} ;$$

$${y}_{i 1}^{′} = \frac{{y}_{i 1} - {y}_{min}}{{y}_{max} - {y}_{min}} ;$$

$${\overline{Y}}_{n 1} = {[ \begin{matrix}{y}_{11}^{′} \\ {y}_{21}^{′} \\ ⋮ \\ {y}_{n 1}^{′}\end{matrix} ]}_{n \times 1} ;$$

wherein x′ᵢⱼ represents the component value of the element in the j^{th} column of the i^{th} sample of the normalized input matrix Xₙₘ in the LLE-SVR model, and xᵢⱼ represents the component value of the element in the j^{th} column of the i^{th} sample of the input matrix Xₙₘ, and xₘᵢₙ represents the minimum of the input matrix Xₙₘ and xₘₐₓ represents the maximum of the input matrix Xₙₘ; X̅ₙₘ represents the normalized matrix of the input matrix Xₙₘ in the LLE-SVR model; the row vector x′ᵢ^{T}=(x′ᵢ₁,x′ᵢ₂, . . . , xᵢₘ) of the i^{th} row of the matrix X̅ₙₘ represents the normalized vectors of the component values of m element(s) contained in the i^{th} sample to be measured; n represents the number of the samples to be measured contained in the standard sample set; m represents the number of the elements in each sample to be measured; y′ᵢ₁ represents the content values of the target elements of the i^{th} sample of the normalized output matrix Yₙ₁ in the LLE-SVR model; yᵢ₁ represents the content values of the target elements of the i^{th} sample of the output matrix Yₙ₁; yₘᵢₙ represents the minimum of the output matrix Yₙ₁; yₘₐₓ represents the maximum of the output matrix Yₙ₁; Y̅ₙ₁ represents the normalized matrix of the output matrix in the LLE-SVR model; i=1, 2, . . . , n, j=1, 2, . . . , m.

Further, the step of searching for neighbor points and calculating a weight value of the neighbor points based on the normalized matrixes of the input matrix and the output matrix, and performing LLE for dimensionality reduction and calculating a component matrix after LLE for dimensionality reduction may comprise:
(1) searching for the neighbor points: in local neighborhood, calculating Euclidean Distance between each normalized sample point x′ᵢⱼ and points of other n-1 samples, and selecting I neighbor points of x′ᵢⱼ;
(2) calculating the weight value W of the neighbor points: calculating the weight value W of each sample point x′ᵢⱼ and I neighbor points of each sample point;

$$W = arg min {\sum }_{i = 1}^{n} ‖ {x}_{ij}^{′} - {\sum }_{p = 1}^{n} {w}_{ip} \cdot {x}_{pq}^{′} ‖ ;$$

wherein, x′ᵢⱼ represents the normalized component value of the element in the j^{th} column of the i^{th} sample, and x′_{pq} represents the normalized component value of the element in the q^{th} column of the p^{th} sample, and wᵢₚ represents the weight value between the sample point and the sample point x′ᵢⱼ and when the sample point x′_{pq} does not belong to the neighbor of the sample point x′ᵢⱼ, wᵢₚ=0; and
(3) mapping the m-dimensional data of each row in the matrix Xₙₘ to the k-dimensional data by nonlinear function mapping: obtaining k principal components by using the local linear embedding method (LLE) for dimensionality reduction, and using the k-dimensional data to reflect the information expressed in the original m-dimensional data, and obtaining the dimensionality-reduced feature Z:

$$Z = arg min {\sum }_{i = 1}^{n} ‖ {z}_{ij} - {\sum }_{p = 1}^{n} {w}_{ip} \cdot {z}_{pq} ‖ ;$$

wherein the dimensionality-reduced feature Z satisfies the following two conditions:

$${\sum }_{i = 1}^{n} {z}_{ij} = 0 ;$$

$$\frac{1}{n} {\sum }_{i = 1}^{n} {z}_{ij} \cdot {z}_{ij}^{T} = 1 ;$$

$$Z = [ \begin{matrix}{Z}_{1}^{T} \\ {Z}_{2}^{T} \\ ⋮ \\ {Z}_{n}^{T}\end{matrix} ] = [ \begin{matrix}{z}_{11} & {z}_{12} & ⋯ & {z}_{1 k} \\ {z}_{21} & {z}_{22} & ⋯ & {z}_{2 k} \\ ⋮ & ⋮ & & ⋮ \\ {z}_{n 1} & {z}_{n 2} & ⋯ & {z}_{nk}\end{matrix} ] ;$$

wherein Z represents the dimensionality-reduced feature matrix, and the dimension k of the dimensionality-reduced feature matrix Z is lower than the dimension n of the original sample (k≤n), and Zᵢⱼ represents the component value of the j^{th} column of the i^{th} sample after LLE for dimensionality reduction, and the row vector Zᵢ^{T}=(zᵢ₁, zᵢ₂, . . . , zᵢₖ of the i^{th} row of the matrix Z represents the component value vector of k elements contained in the i^{th} sample to be measured i=1, 2, . . . , n, j=1, 2, . . . , k.

Further, the fourth step of performing nonlinear function mapping and constructing a classification hyperplane, and introducing a penalty factor and a slack variable for constraint, and training the LLE-SVR model via parameter optimization to quantitatively predict the element content may comprise:
1) mapping the k-dimensional element component value data from the low-dimensional nonlinear separable space to a high-dimensional linear separable feature space, and constructing a classification hyperplane in this high-dimensional linear separable feature space:

hₚ[(w·φ(xₚ))+b]−1≥0;

wherein hₚ represent the class marker under p dimension, and when located above the hyperplane w·φ(xₚ)+b it is defined hₚ=1; when located below the hyperplane w·φ(xₚ)+b , it is defined hₚ=−1; p=1, 2, . . . , k, k≤m; w represents the weight value vector of the feature, and b represents the bias; xₚ represents the element component value vector after the dimensionality reduction of the sample to be measured into p dimensions via PCA, and φ(xₚ)represents a nonlinear mapping function mapping the data xₚ to the high-dimensional linear separable feature space;
2) introducing the penalty factor C and the slack variable ξₚ for constraint and converting the classification hyperplane problem into a quadratic programming model:

$$\{ \begin{matrix}min 1 / 2 {‖ w ‖}^{2} + C {\sum }_{p = 1}^{k} {ξ}_{p} \\ s . t . {h}_{p} ( ( w \cdot φ ( {x}_{p} ) ) + b \geq 1 - {ξ}_{p} \\ {ξ}_{p} \geq 0 , p = 1 , 2 , ... , k\end{matrix} ;$$

3) training the LLE-SVR model by parameter optimization using the cross-validation method based on grid search: obtaining the optimal parameter penalty factor C and the optimal slack variable ξₚ by iteratively searching for the optimal parameters; and
4) introducing a Lagrangian multiplier αₚ and a kernel function K to calculate the minimum classification hyperplane satisfying the quadratic programming model, which is prediction result ŷ′ᵢ , of the target element content, and the calculation formula for predicting the target element content of any i^{th} sample to be measured being:

$${\hat{y}}_{i 1}^{′} = {\sum }_{p = 1}^{k} {α}_{p} {h}_{p} K + b ;$$

$${\hat{Y}}_{n 1}^{′} = {[ \begin{matrix}{\hat{y}}_{11}^{′} \\ {\hat{y}}_{21}^{′} \\ ⋮ \\ {\hat{y}}_{n 1}^{′}\end{matrix} ]}_{n \times 1} .$$

Further, the fifth step of denormalizing the normalized output matrix of the target element content to obtain the denormalized output matrix, and calculating the coefficient of determination to evaluate the predicting effect of the LLE-SVR model, may comprise:
(1) denormalizing the normalized output matrix Ŷ′ₙ₁ of the target element content to obtain the denormalized output matrix Ŷₙ₁;

$${\hat{y}}_{i 1} = {\hat{y}}_{i}^{′} ( {\hat{y}}_{max}^{′} - {\hat{y}}_{min}^{′} ) + {\hat{y}}_{min}^{′} ;$$

$${\hat{Y}}_{n 1} = {[ \begin{matrix}{\hat{y}}_{11} \\ {\hat{y}}_{21} \\ ⋮ \\ {\hat{y}}_{n 1}\end{matrix} ]}_{n \times 1} ;$$

wherein ŷᵢ₁ represents the prediction value of the target element content of the i^{th} sample of the denormalized output matrix Ŷₙ₁, and y′ₘᵢₙ represent the minimum of the output matrix , and yₘₐₓ represents the maximum of the output matrix Ŷ′ₙ₁; i=1,2, . . . , n; and

(2) comparing the predicted target element content ŷᵢ₁ with the true target element content yᵢ₁, and calculating the coefficient of determination R²:

$${R}^{2} = 1 - \frac{{\sum }_{i = 1}^{n} {( {y}_{i 1} - {\hat{y}}_{i 1} )}^{2}}{{\sum }_{i = 1}^{n} {( {y}_{i 1} - {\overline{y}}_{i 1} )}^{2}} ;$$

$${\overline{y}}_{i 1} = \frac{{\sum }_{i = 1}^{n} {y}_{i 1}}{n} ;$$

wherein yᵢ₁ represents the true value of the target element content of the i^{th} sample, and ŷᵢ₁ represents the prediction value of the target element content of the i^{th} sample, and y̅ᵢ₁ represents the average of the true value of the target element content of the i^{th} sample to be measured; i=1, 2, . . . , n.

Another object of the present disclosure is to provide a computer device comprising a storage storing a computer program, and a processor, and when the computer program is executed by the processor, the steps of the method of XRF quantitative analysis of heavy metal elements based on LLE-SVR is performed by the processor.

Another object of the present disclosure is to provide a computer-readable storage medium storing a computer program, and when the computer program is executed by the processor, the steps of the method of XRF quantitative analysis of heavy metal elements based on LLE-SVR is performed by the processor.

Another object of the present disclosure is to provide an information data processing terminal used to implement the method of XRF quantitative analysis of heavy metal elements based on LLE-SVR.

Combined with the technical problems to be solved and the above technical solutions, the advantages and positive effects of the claimed technical solution in the present disclosure are as follows:

First, The local linear embedding (LLE) algorithm used in the present disclosure is based on sparse matrix feature decomposition, with relatively low computational complexity and being easy to implement, and the local features of the sample are maintained during dimensionality reduction.

The support vector regression (SVR) method used in the present disclosure is a novel method with a solid theoretical foundation, which is suitable for few-shot learning. It maps vectors into a higher-dimensional space where a maximum-margin hyperplane is established. Two parallel hyperplanes are built on both sides of the hyperplane that separates the data, and the separating hyperplane maximizes the distance between the two parallel hyperplanes, so that the element content can be predicted more accurately. The SVR method is insensitive to outliers, which can effectively grasp key samples and exhibits high robustness and excellent generalization ability.

In the present disclosure, the combined algorithm of the Locally Linear Embedding (LLE) and Support Vector Regression (SVR) is applied to the quantitative analysis of element contents. First, the complex redundant data is eliminated by LLE for dimensionality reduction, and then the SVR method is used for content prediction, the combination of which solves the problem of spectral line interference, such as overlapping peaks and escape peaks, and provides a new means for improving the content detection accuracy of heavy metal elements.

In the application and implementation, the content of element Pb in 57 nationally standard soil samples was predicted with high prediction accuracy, which provides an innovative method and technical support for the detection of heavy metal elements in soil.

Second, the quantitative analysis process of the present disclosure is simple, scientific and reasonable, with high prediction accuracy and intuitive results, and is easy to understand; the quantitative analysis method of the present disclosure has the characteristics of high detection precision and high prediction accuracy, and solves the problems of inaccuracy and peak overlapping interference between elements from traditional instrument measurement methods by establishing the relationship between element component value and element content, reducing the influence of environmental background, and realizing accurate prediction of the element content contained in the analytes.

Third, the present disclosure proposes an method of XRF quantitative analysis of heavy metal elements based on LLE-SVR, which improves the accuracy of element content prediction, i.e., it can more accurately analyze the geographical location of the pollution source, provide effective support for early treatment of soil environmental pollution, and directly reduce the cost of the soil treatment market by about 15%;

For a long time, people have never stopped research on the estimation of heavy metal elements, but the low accuracy of the system has always been a challenge in the industry, and the present disclosure well improves the accuracy and stability of the system by the method of XRF quantitative analysis of heavy metal elements based on LLE-SVR.

### BRIEF DESCRIPTION OF THE DRAWINGS

FIG. 1 is a schematic diagram of a method of XRF quantitative analysis of heavy metal elements based on LLE-SVR provided by an embodiment of the present disclosure;

FIG. 2 is a flow chart of a method of XRF quantitative analysis of heavy metal elements based on LLE-SVR provided by an embodiment of the present disclosure;

FIG. 3 is a spectrogram of a standard soil sample provided by an embodiment of the present disclosure;

FIG. 4 is a plot of the prediction result of the content of element Co in soil based on the LLE-SVR model provided by an embodiment of the present disclosure.

### DETAILED DESCRIPTION

In order to make the objectives, technical solution and advantages of the present disclosure clearer, the present disclosure will be further explained in detail with reference to the following examples. It should be understood that the specific examples described herein are only for explaining the present disclosure, but not for limiting the present disclosure.

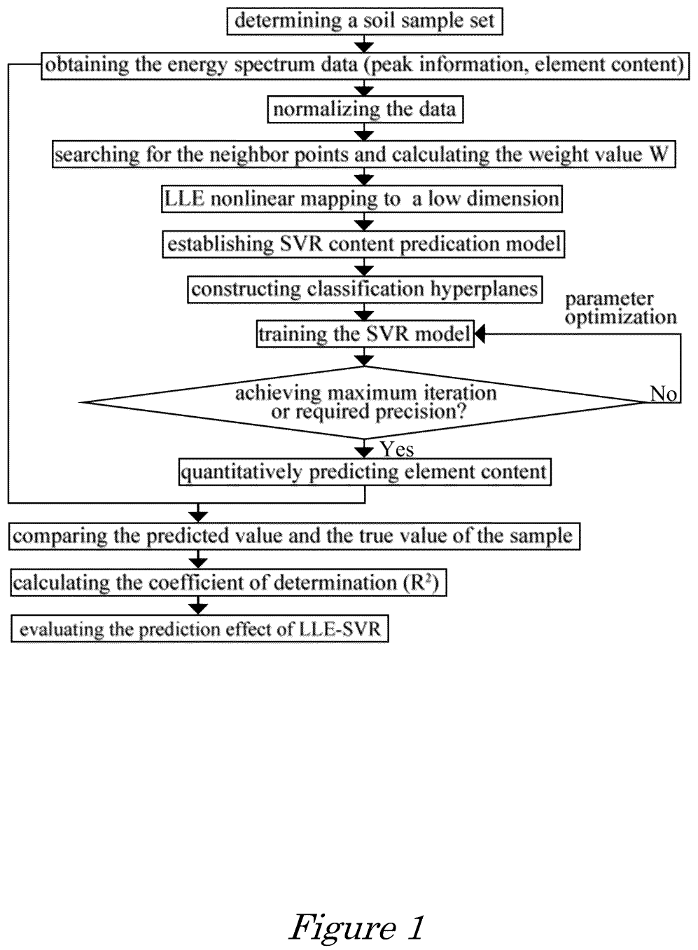

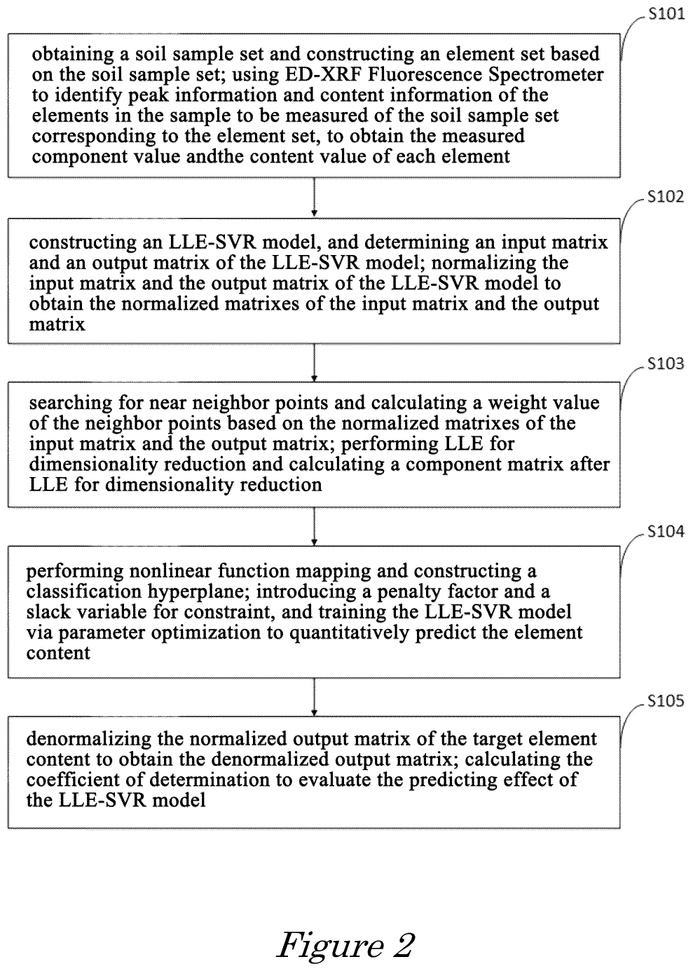

As shown in FIGS. 1-2, an example of the present disclosure provides a method of XRF quantitative analysis of heavy metal elements based on LLE-SVR, comprising, in some embodiments, the following procedures and/or steps.

S101, a soil sample set was obtained and an element set was constructed based on said soil sample set; the ED-XRF fluorescence spectrometer was used to identify the peak information and content information of the elements in the sample to be measured in the soil sample set corresponding to that in the element set, to obtain the measured component value and content value of each element.

S102, an LLE-SVR model was constructed, and the input matrix and the output matrix of the LLE-SVR model was determined; the input matrix and the output matrix of the LLE-SVR model was normalized to obtain the normalized matrixes of the input matrix and the output matrix.

S103, the neighbor points were searched for based on the normalized matrixes of the input matrix and the output matrix and the weight values of the neighbor points were calculated; the LLE was performed for dimensionality reduction, and the component matrix after LLE for dimensionality reduction was calculated.

S104, the nonlinear function mapping was performed and the classification hyperplane was constructed; a penalty factor and a slack variable were introduced for constraint, and the LLE-SVR model was trained by parameter optimization for quantitative prediction of element content.

S105, the normalized output matrix of the target element content was denormalized to obtain a denormalized output matrix; the coefficient of determination was calculated to evaluate the prediction effect of the LLE-SVR model.

An example of the present disclosure provides an XRF quantitative analysis method of heavy elements based on LLE-SVR, comprising, for example, the following steps:

Step 1: a soil sample set was determined, and it is supposed that the soil sample set had n samples, which were sample 1, sample 2, . . . , sample 57, respectively. All elements which can be identified by the spectrometer was taken out to constitute an element set A contained in the soil sample, and finally, 57 element sets A1-A57 was obtained, i.e., the union set of A1-A57 is the element set A contained in the soil sample set, and the element set A is the element library of element Nos. 12-92 in periodic table.

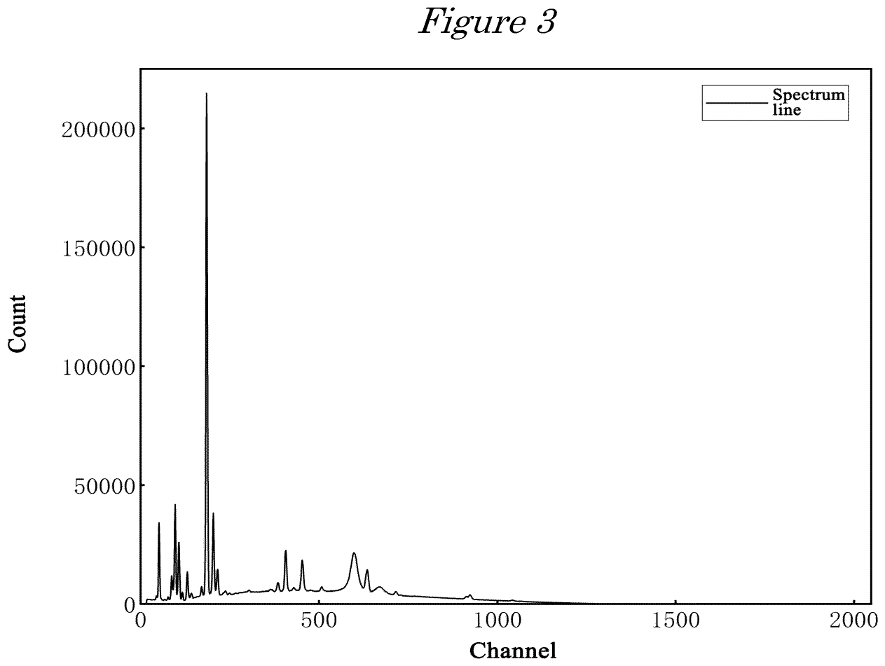

Step 2: 57 nationally standard samples were adopted as the standard samples comprising three types of standard substances including GSS series, GBW series and GSD series. GSS Series Reference Material of Chemical Composition of Soil comprises GSS-1, GSS-2, GSS-3, GSS-4, GSS-5, GSS-6, GSS-7, GSS-8, GSS-9, GSS-10, GSS-11, GSS-12, GSS-13, GSS-14, GSS-15, GSS-16, GSS-17, GSS-18, GSS-19, GSS-20, GSS-21, GSS-22, GSS-23, GSS-24, GSS-25, GSS-26, GSS-27, GSS-32, and GBW Series Reference Material of Chemical Composition of Soil comprises GBW0070003, GBW0070004, GBW0070005, and GSD Series Reference Materials for the Chemical Composition of Stream Sediments comprises GSD-2a, GSD-3, GSD-5a, GSD-9, GSD-11, GSD-12, GSD-14, GSD-15, GSD-16, GSD-17, GSD-18, GSD-19, GSD-20, GSD-21, GSD-22, GSD-23, GSD-25, GSD-26, GSD-27, GSD-28, GSD-29, GSD-30, GSD-31, GSD-32, GSD-33. The XRF spectrogram of the samples and the component value X and the content value Y of the element contained in the sample can be obtained simultaneously by XRF Fluorescence Spectrometer, and the XRF spectrogram of the standard soil sample was shown in FIG. 3.

Step 3: In the element set A, the union set of the studied target elements and their corresponding interfering elements was taken as an input variable of the LLE-SVR model, and the example mainly studied harmful elements of soil which were: 23 (V), 24 (Cr), 25 (Mn), 27 (Co), 29 (Cu), 30 (Zn), 48 (Cd), 82 (Pb), in a total of eight elements. 57 standard soil samples were taken as examples to record the component content of the target elements. The matrix of the measured component values composed of the target elements and their interfering elements was taken as the input of the LLE-SVR model, and the matrix of the target element Pb content was used as the output of the LLE-SVR model, and the details of the interfering elements are shown in Table 1.

TABLE 1
Main interfering elements of target elements

| Target element | Main interfering element |
|---|---|
| V | Ti, As, K |
| Cr | Ni, Se, Fe |
| Mn | Fe, Ni, Nb |
| Co | Fe, V, Zn, Sr |
| Cu | Fe, Ni, P, Co, Mn |
| Zn | Fe, Ni, Cu, V, Cr, Co |
| Cd | Ag, Zn, Sb, Pb, K |
| Pb | Fe, Ni |

Taking element Co as the target element for content prediction as an example, the input of the LLE-SVR model was a 57×19 component data matrix, i.e., a matrix containing 57 samples, and each sample consists of the component values of 19 elements (the union set of all target elements and their corresponding interfering elements). Similarly, the output of the LLE-SVR model is a 57×1 component data matrix, i.e. a matrix of 57 samples, each consisting of a content value of a single target element (Co).

Step 4: the XRF spectrum data was normalized: the input matrix Xₙₘ and the output matrix Yₙ₁ were normalized to obtain the normalized matrixes X̅ₙₘ and y̅ₙₘ. The process of normalizing the matrixes was as follows:

$${x}_{ij}^{′} = \frac{{x}_{ij} - {x}_{min}}{{x}_{max} - {x}_{min}} \qquad ( 1 )$$

$${\overline{X}}_{nm} = [ \begin{matrix}{x}_{1}^{{′}^{T}} \\ {x}_{2}^{{′}^{T}} \\ ⋮ \\ {x}_{n}^{{′}^{T}}\end{matrix} ] = {[ \begin{matrix}{x}_{11}^{′} & {x}_{12}^{′} & ⋯ & {x}_{1 m}^{′} \\ {x}_{21}^{′} & {x}_{22}^{′} & ⋯ & {x}_{2 m}^{′} \\ ⋮ & ⋮ & & ⋮ \\ {x}_{n 1}^{′} & {x}_{n 2}^{′} & ⋯ & {x}_{nm}^{′}\end{matrix} ]}_{n \times m} \qquad ( 2 )$$

$${y}_{i 1}^{′} = \frac{{y}_{i 1} - {y}_{min}}{{y}_{max} - {y}_{min}} \qquad ( 3 )$$

$${\overline{Y}}_{n 1} = {[ \begin{matrix}{y}_{11}^{′} \\ {y}_{21}^{′} \\ ⋮ \\ {y}_{n 1}^{′}\end{matrix} ]}_{n \times 1} \qquad ( 4 )$$

wherein i=1, 2, . . . , n, j=1, 2, . . . , m. The row vector x′ᵢ^{T}=(x′ᵢ₁, x′ᵢ₂, . . . , x′ᵢₘ)of the i^{th} row of the matrix X̅ₙₘ represented the normalized component value vector of m element(s) contained in the i^{th} sample to be measured, and xₘᵢₙ represented the minimum of the input matrix Xₙₘ, and xₘₐₓ represented the maximum of the input matrix Xₙₘ, and yₘᵢₙ represented the minimum of the output matrix Yₙ₁, and yₘₐₓ represented the maximum of the output matrix Yₙ₁. xᵢⱼ represented the component value of the element in the j^{th} column of the i^{th} sample of the input matrix Xₙₘ, and x′ᵢⱼ represented the normalized component value of the element in the j^{th} column of the i^{th} sample of the input matrix XYₙₘ. yᵢ₁ represented the content value of the target element of the i^{th} sample of the output matrix Yₙ₁, and y′ᵢ₁ represented the normalized content value of the target element of the i^{th} sample of the output matrix Yₙ₁.

Step 5: the neighbor points were searched for: in local neighborhood, the Euclidean Distance between each normalized sample point x′ᵢⱼ and the points of other n-1 samples were calculated, and I neighbor points of x′ᵢⱼ were selected;

Step 6: the weight value W of the neighbor point was calculated: the weight value W of each sample point x′ᵢⱼ and its I neighbor points, and the calculation formula for the weight value W was as follows

$$W = arg min {\sum }_{i = 1}^{n} ‖ {x}_{ij}^{′} - {\sum }_{p = 1}^{n} {w}_{ip} \cdot {x}_{pq}^{′} ‖ \qquad ( 5 )$$

wherein x′ᵢⱼ represented the normalized component value of the element in the j^{th} column of the i^{th} sample, and x′_{pq} represented the normalized component value of the element in the q^{th} column of the p^{th} sample, and wᵢₚ represented the weight value between the sample point x′ᵢⱼ and the sample point x′_{pq}, and when the sample point x′_{pq} did not belong to the neighbor of the sample point x′ᵢⱼ, wᵢₚ =0;

Step 7: the LLE was used for dimensionality reduction: according to the reconstruction weight value, all sample data points were mapped into the low-dimensional space to obtain the low-dimensional output Z, and the local linear features in the high-dimensional space were maintained as much as possible to minimize the reconstruction error function. The m-dimensional data of each row in the matrix Xₙₘ was mapped to the k-dimensional data by nonlinear function mapping, i.e., obtaining k principal components after the local linear embedding method (LLE) for dimensionality reduction, and using the k-dimensional data to reflect the information expressed in the original m-dimensional data, and the dimensionality-reduced feature was:

$$Z = arg min {\sum }_{i = 1}^{n} ‖ {z}_{ij} - {\sum }_{p = 1}^{n} {w}_{ip} \cdot {z}_{pq} ‖ \qquad ( 6 )$$

provided that:

$${\sum }_{i = 1}^{n} {z}_{ij} = 0 \qquad ( 7 )$$

$$\frac{1}{n} {\sum }_{i = 1}^{n} {z}_{ij} \cdot {z}_{ij}^{T} = 1 \qquad ( 8 )$$

$$Z = [ \begin{matrix}{Z}_{1}^{T} \\ {Z}_{2}^{T} \\ ⋮ \\ {Z}_{n}^{T}\end{matrix} ] = [ \begin{matrix}{z}_{11} & {z}_{12} & … & {z}_{1 k} \\ {z}_{21} & {z}_{22} & … & {z}_{2 k} \\ ⋮ & ⋮ & & ⋮ \\ {z}_{n 1} & {z}_{n 2} & … & {z}_{nk}\end{matrix} ] \qquad ( 9 )$$

wherein i=1, 2, . . . , n, j=1, 2, . . . , k and Z represented the dimensionality-reduced feature matrix, and the dimension k of the dimensionality-reduced feature matrix Z was lower than the dimension n of the original sample (k≤n). A′ᵢ^{T} represented the row vector of the i^{th} row of the normalized matrix X̅, and zᵢⱼ represented the component value of the j^{th} column of the i^{th} sample after the LLE for dimensionality reduction. The row vector Zᵢ^{T}=(zᵢ₁, zᵢ₂, . . . , zᵢₖ of the i^{th} row of the matrix Z represented the component value vector of k elements contained in the i^{th} sample to be measured.

Step 8: the k-dimensional element component value data was mapped from the low-dimensional nonlinear separable space to a high-dimensional linear separable feature space, and a classification hyperplane was constructed in this high-dimensional linear separable feature space:

hₚ[(w·φ(xₚ))+b]−1≥0 (10)
wherein p=1, 2, . . . , k , k≤m, and hₚ represented the class marker under p dimension, and when located above the hyperplane w·φ(xₚ)+b it was defined hₚ=1; when located below the hyperplane w·φ(xₚ)+b, it was defined hₚ=−1; w represented the weight vector of the feature, and b represented the bias; xₚ represented the element component value vector after the dimensionality reduction of the sample to be measured into p dimensions via PCA, and φ(xₚ)represented a nonlinear mapping function mapping the data xₚ to the high-dimensional linear separable feature space; to simplify the formula, the subscript i in xₚ (different samples to be measured corresponding to different xₚ ) was omitted;

Step 9: a penalty factor ξₚ and a slack variable were introduced for constraint and the classification hyperplane problem was converted into a quadratic programming model:

$$\{ \begin{matrix}min 1 / 2 {‖ w ‖}^{2} + C {\sum }_{p = 1}^{k} {ξ}_{p} \\ s . t . {h}_{p} ( ( w \cdot φ ( {x}_{p} ) ) + b \geq 1 - {ξ}_{p} \\ {ξ}_{p} \geq 0 , p = 1 , 2 , … , k\end{matrix} \qquad ( 11 )$$

Step 10: the LLE-SVR model was trained by searching for parameter optimization using the cross-validation method based on grid search. The optimal parameter penalty factor C and the optimal slack variable ξₚ were obtained by iteratively searching for the optimal parameters. The Lagrangian multiplier αₚ and the kernel function K was introduced to solve the formula (11), and the minimum classification hyperplane satisfying the required precision was the prediction result ŷ′ᵢ, of the target element content, and the calculation formula for predicting the target element content of any i^{th} sample to be measured was:

$${\hat{y}}_{i 1}^{′} = {\sum }_{p = 1}^{k} {α}_{p} {h}_{p} K + b \qquad ( 12 )$$

$${\hat{Y}}_{n 1}^{′} = {[ \begin{matrix}{\hat{y}}_{11}^{′} \\ {\hat{y}}_{21}^{′} \\ ⋮ \\ {\hat{y}}_{n 1}^{′}\end{matrix} ]}_{n \times 1} \qquad ( 13 )$$

Step 11: the XRF spectrum data was denormalized; the normalized output matrix Ŷ′ₙ₁ of the target element content was denormalized to obtain the denormalized output matrix Ŷ′ₙ₁. The process of denormalizing the matrix was as follows:

$${\hat{y}}_{il} = {\hat{y}}_{1}^{′} ( {\hat{y}}_{max}^{′} - {\hat{y}}_{min}^{′} ) + {\hat{y}}_{min}^{′} \qquad ( 14 )$$

$${\hat{Y}}_{n 1}^{′} = {[ \begin{matrix}{\hat{y}}_{11}^{′} \\ {\hat{y}}_{21}^{′} \\ ⋮ \\ {\hat{y}}_{n 1}^{′}\end{matrix} ]}_{n \times 1} \qquad ( 15 )$$

wherein, i=1, 2, . . . , ŷᵢ₁ represents the prediction value of the target element content of the i^{th} sample of the denormalized output matrix Ŷₙ₁, and y′ₘᵢₙ represented the minimum value of the output matrix Ŷ′ₙ₁, and y′ₘₐₓ represented the maximum value of the output matrix Ŷ′ₙ₁.

Step 12: the predicted target element content ŷᵢ₁ was compared with the true target element content yᵢ₁, and the coefficient of determination (R²) was calculated. The calculation formula for R² was as follows, respectively:

$${R}^{2} = 1 - \frac{{\sum }_{i = 1}^{n} {( {y}_{i 1} - {\hat{y}}_{i 1} )}^{2}}{{\sum }_{i = 1}^{n} {( {y}_{i 1} - {\overline{y}}_{i 1} )}^{2}} \qquad ( 16 )$$

$${\overline{y}}_{i 1} = \frac{{\sum }_{i = 1}^{n} {y}_{i 1}}{n} \qquad ( 17 )$$

wherein i=1, 2, . . . , n. yᵢ₁ represented the true value of the target element content of the i^{th} sample, and ŷᵢ₁ represented the prediction value of the target element content of the i^{th} sample, and y̅ᵢ₁ represented the average of the true value of the target element content of the i^{th} sample to be measured.

The method of XRF quantitative analysis of heavy metal elements based on LLE-SVR provided by the example of the present disclosure was applied with a computer device, which comprised a storage storing a computer program, and a processor, and when the computer program was executed by the processor, the processor performed the steps of the method of XRF quantitative analysis of heavy metal elements based on LLE-SVR.

The method of XRF quantitative analysis of heavy metal elements based on LLE-SVR provided by the example of the present disclosure was applied with a computer-readable storage medium storing a computer program, and when the computer program was executed by the processor, the processor performed the steps of the method of XRF quantitative analysis of heavy metal elements based on LLE-SVR.

The method of quantitative analysis of XRF heavy metal elements based on LLE-SVR provided by the example of the present disclosure was applied with an information data processing terminal, which is used to realize the method of quantitative analysis of XRF heavy metal elements based on LLE-SVR.

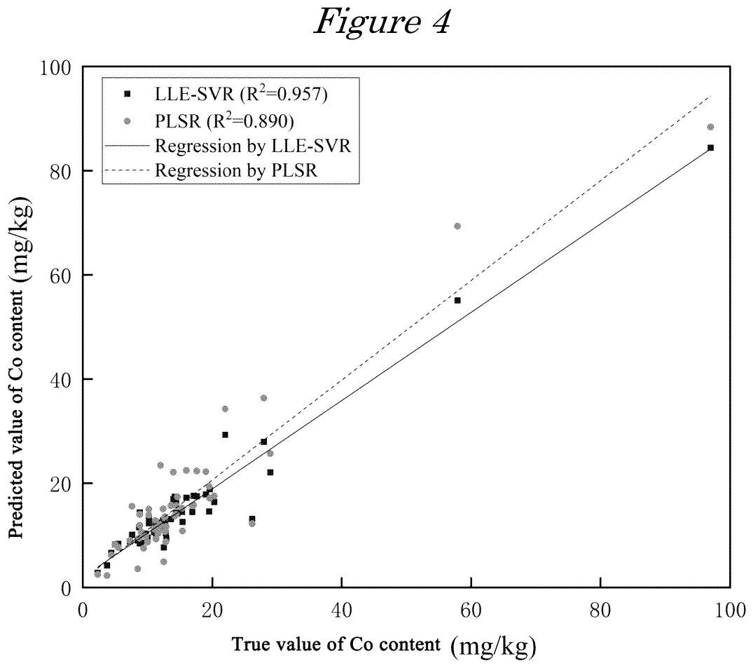

Taking the heavy metal element Co as an example, comparing the resulted coefficients of determination R 2 of the standard soil sample element between the traditional partial least squares regression (PLSR) method and the method based on the LLE-SVR, and the content prediction results were shown in FIG. 4. Compared to the partial least squares regression (PLSR) method, the prediction results of XRF quantitative analysis of heavy metal elements based on LLE-SVR of the present disclosure were more in conformity with the true results of element content. It was shown that the LLE-SVR algorithm of the present disclosure effectively solved the problem of the overlapping spectral lines, improved the accuracy of the quantitative analysis results of elements, and reflected the superiority of the method of the present disclosure.

It should be noted that, the examples of the present disclosure may be implemented by hardware, software, or a combination of software and hardware. The part of hardware can be implemented using specialized logic; the part of software can be stored in memory and executed by an appropriate instruction execution system, such as a microprocessor or a specialized design hardware. Those skilled in the art may understand that the above devices and methods may be implemented using computer executable instructions and/or contained in processor control code, for example providing such code on a carrier medium, such as a disk, CD or DVD-ROM, a programmable memory such as a read-only memory (firmware), or a data carrier such as an optical or an electronic signal carrier. The devices and modules thereof of the present disclosure may be implemented by a hardware circuit, such as a very large scale integrated circuit or a gate array, such as a semiconductor, for example, a logic chip, a transistor, or a programmable hardware device such as a field programmable gate array, programmable logic device, etc., or may be implemented with software executed by various types of processors, or may be implemented by a combination of the above hardware circuits and software, such as firmware.

The above merely describes the specific embodiments of the present disclosure, which is not intended to limit the scope of protection of the present disclosure. Any modifications, equivalent substitutions and improvements made within the spirit and principle of the present disclosure by those skilled in the art according to the disclosed technical scope should be included in the protection scope of the present disclosure.

What is claimed is:

1. A method of XRF quantitative analysis of heavy metal elements based on LLE-SVR, wherein the method comprises:
establishing the relationship between peak information and element content by using Local Linear Embedding For Dimensionality Reduction and Support Vector Regression Predictive Algorithms based on machine learning, to quantitatively analyze of the content information of elements contained in substances.

2. The method of XRF quantitative analysis of heavy metal elements based on LLE-SVR according to claim 1, wherein the method comprises the following steps of:
a first step of obtaining a soil sample set and constructing an element set based on the soil sample set, and using ED-XRF Fluorescence Spectrometer to identify peak information and content information of the elements in the sample to be measured of the soil sample set corresponding to the element set, to obtain the measured component value and the content value of each element;
a second step of constructing an LLE-SVR model, and determining an input matrix and an output matrix of the LLE-SVR model, and normalizing the input matrix and the output matrix of the LLE-SVR model to obtain the normalized matrixes of the input matrix and the output matrix;
a third step of searching for neighbor points and calculating a weight value of the neighbor points based on the normalized matrixes of the input matrix and the output matrix, and performing LLE for dimensionality reduction and calculating a component matrix after LLE for dimensionality reduction;
a fourth step of performing nonlinear function mapping and constructing a classification hyperplane, and introducing a penalty factor and a slack variable for constraint, and training the LLE-SVR model via parameter optimization to quantitatively predict the element content; and
a fifth step of denormalizing the normalized output matrix of the target element content to obtain the denormalized output matrix, and calculating the coefficient of determination to evaluate the predicting effect of the LLE-SVR model.

3. The method of XRF quantitative analysis of heavy metal elements based on LLE-SVR according to claim 2, wherein the first step of obtaining the soil sample set and constructing the element set based on the soil sample set comprises:
obtaining a standard sample set, and identifying all elements of all samples to be measured in the standard sample set by using ED-XRF Fluorescence Spectrometer, to obtain an element set A containing all elements of all samples to be measured in the standard sample set;
where said all elements are element Nos. 12-92 in periodic table;
the input matrix of the LLE-SVR model in the second step is a matrix composed of the measured component values of all target elements of the element set A and their corresponding interfering elements;
the output matrix of the LLE-SVR model is a matrix composed of the measured content values of the target elements;
the target elements are the elements to be quantitatively analyzed; the interfering elements are the elements that interfere with the target elements;
the matrix of the measured component values is a matrix composed of the component values of m element(s) contained in each of n sample(s) to be measured; the first column of the matrix of the measured component values is the measured component values of a single target element, and the remaining m-1 column(s) of the matrix of the measure component values is composed of the measured component values of other target elements and the interfering elements corresponding to all target elements; and
the matrix of the measured content values is a matrix composed of the concentration values of the single target element contained in each of n samples to be measured.

4. The method of XRF quantitative analysis of heavy metal elements based on LLE-SVR according to claim 2, wherein the second step of normalizing the input matrix and the output matrix of the LLE-SVR model comprises:
normalizing the input matrix and the output matrix to obtain the normalized matrixes:
$${x}_{ij}^{′} = \frac{{x}_{ij} - {x}_{min}}{{x}_{max} - {x}_{min}} ;$$
$${\overline{X}}_{nm} = [ \begin{matrix}{x}_{1}^{′ T} \\ {x}_{2}^{′ T} \\ ⋮ \\ {x}_{n}^{′ T}\end{matrix} ] = {[ \begin{matrix}{x}_{11}^{′} & {x}_{12}^{′} & … & {x}_{1 m}^{′} \\ {x}_{21}^{′} & {x}_{22}^{′} & … & {x}_{2 m}^{′} \\ ⋮ & ⋮ & & ⋮ \\ {x}_{n 1}^{′} & {x}_{n 2}^{′} & … & {x}_{nm}^{′}\end{matrix} ]}_{n \times m} ;$$
$${y}_{i 1}^{′} = \frac{{y}_{i 1} - {y}_{min}}{{y}_{max} - {y}_{min}} ;$$
$${\overline{Y}}_{n 1}^{′} = {[ \begin{matrix}{\hat{y}}_{11}^{′} \\ {\hat{y}}_{21}^{′} \\ ⋮ \\ {\hat{y}}_{n 1}^{′}\end{matrix} ]}_{n \times 1} ;$$
wherein x′ᵢⱼ represents the component value of the element in the j^{th} column of the i^{th} sample of the normalized input matrix Xₙₘ in the LLE-SVR model, and xᵢⱼ represents the component value of the element in the j^{th} column of the i^{th} sample of the input matrix Xₙₘ, and xₘᵢₙ represents the minimum of the input matrix Xₙₘ, and xₘₐₓ represents the maximum of the input matrix Xₙₘ; X̅ₙₘ represents the normalized matrix of the input matrix Xₙₘ in the LLE-SVR model; the row vector x′ᵢ^{T}(x′ᵢ₁, x′ᵢ₂, . . . , x′ᵢₘ) of the i^{th} row of the matrix X̅ₙₘ represents the normalized vector of the component values of m element(s) contained in the i^{th} sample to be measured; n represents the number of the samples to be measured contained in the standard sample set; m represents the number of the elements in each sample to be measured; y′ᵢ₁represents the content values of the target elements of the i^{th} sample of the normalized output matrix Yₙ₁in the LLE-SVR model; yᵢ₁represents the content values of the target elements of the i^{th} sample of the output matrix Yₙ₁; yₘᵢₙ represents the minimum of the output matrix Yₙ₁; yₘₐₓ represents the maximum of the output matrix Yₙ₁; Ŷₙ₁ represents the normalized matrix of output matrix in the LLE-SVR model; i=1, 2, . . . , n, j=1, 2, . . . , m

5. The method of XRF quantitative analysis of heavy metal elements based on LLE-SVR according to claim 2, wherein the step of searching for neighbor points and calculating a weight value of the neighbor points based on the normalized matrixes of the input matrix and the output matrix, and performing LLE for dimensionality reduction and calculating a component matrix after LLE for dimensionality reduction comprises:
(1) searching for the neighbor points: in local neighborhood, calculating Euclidean Distance between each normalized sample point x′ᵢⱼ and the points of other n-1 samples, and selecting I neighbor points of x′ᵢⱼ;
(2) calculating the weight value W of the neighbor points: calculating the weight value W of each sample point x′ᵢⱼ and I neighbor points of each sample point;
$$W = arg min {\sum }_{i = 1}^{n} ‖ {x}_{ij}^{′} - {\sum }_{p = 1}^{n} {w}_{ip} \cdot {x}_{pq}^{′} ‖ ;$$
wherein, x′ᵢⱼ represents the normalized component value of the element in the j^{th} column of the i^{th} sample, and x′_{pq} represents the normalized component value of the element in the q^{th} column of the p^{th} sample, and wᵢₚ represents the weight value between the sample point x′ᵢⱼ and the sample point x′_{pq}, and when the sample point x′_{pq} does not belong to the neighbor of the sample point x′ᵢⱼ, wᵢₚ=0; and
(3) mapping the m-dimensional data of each row in the matrix Xₙₘ to the k-dimensional data by nonlinear function mapping: obtaining k principal components by using the local linear embedding method (LLE) for dimensionality reduction, and using the k-dimensional data to reflect the information expressed in the original m-dimensional data, and obtaining the dimensionality-reduced feature Z:
$$Z = arg min {\sum }_{i = 1}^{n} ‖ {z}_{ij} - {\sum }_{p = 1}^{n} {w}_{ip} \cdot {z}_{pq} ‖ ;$$
wherein the dimensionality-reduced feature Z satisfies the following two conditions:
$${\sum }_{i = 1}^{n} {z}_{ij} = 0 ;$$
$$\frac{1}{n} {\sum }_{i = 1}^{n} {z}_{ij} \cdot {z}_{ij}^{T} = 1 ;$$
$$Z = [ \begin{matrix}{Z}_{1}^{T} \\ {Z}_{2}^{T} \\ ⋮ \\ {Z}_{n}^{T}\end{matrix} ] = [ \begin{matrix}{z}_{11} & {z}_{12} & … & {z}_{1 k} \\ {z}_{21} & {z}_{22} & … & {z}_{2 k} \\ ⋮ & ⋮ & & ⋮ \\ {z}_{n 1} & {z}_{n 2} & … & {z}_{nk}\end{matrix} ] ;$$
wherein Z represents the dimensionality-reduced feature matrix, and the dimension k of the dimensionality-reduced feature matrix Z is lower than the dimension n of the original sample (k≤n), and zᵢⱼ represents the component value of the j^{th} column of the i^{th} sample after LLE for dimensionality reduction, and the row vector Zᵢ^{T}=(zᵢ₁, zᵢ₂, . . . , zᵢₖ) of the i^{th} row of the matrix Z represents the component value vector of k elements contained in the i^{th} samples to be measured i=1, 2, . . . , n, j=1, 2, . . . , k.

6. The method of XRF quantitative analysis of heavy metal elements based on LLE-SVR according to claim 2, wherein the forth step of performing nonlinear function mapping and constructing a classification hyperplane, and introducing a penalty factor and a slack variable for constraint, and training the LLE-SVR model via parameter optimization to quantitatively predict the element content comprises:
1) mapping the k-dimensional element component value data from the low-dimensional nonlinear separable space to a high-dimensional linear separable feature space, and constructing a classification hyperplane in this high-dimensional linear separable feature space:
hₚ[(w·φ(xₚ))+b]−1≥0;
wherein ₚ represent the class marker under p dimension, and when located above the hyperplane w·φ(xₚ)+b it is defined hₚ=1; when located below the hyperplane w·φ(xₚ)+b, it is defined hₚ=−1; p=1, 2, . . . , k, k≤m; w represents the weight value vector of the feature, and b represents the bias; xₚ represents the element component value vector after the dimensionality reduction of the sample to be measured into p dimensions via PCA, and φ(xₚ)represents a nonlinear mapping function mapping the data xₚ to the high-dimensional linear separable feature space;
2) introducing the penalty factor ξₚ and the slack variable for constraint and converting the classification hyperplane problem into a quadratic programming model:
$$\{ \begin{matrix}min 1 / 2 {‖ w ‖}^{2} + C {\sum }_{p = 1}^{k} {ξ}_{p} \\ s . t . {h}_{p} ( ( w \cdot φ ( {x}_{p} ) ) + b \geq 1 - {ξ}_{p} \\ {ξ}_{p} \geq 0 , p = 1 , 2 , … , k\end{matrix} ;$$
3) training the LLE-SVR model by parameter optimization using the cross-validation method based on grid search: obtaining the optimal parameter penalty factor and the optimal slack variable by iteratively searching for the optimal parameters; and
4) introducing a Lagrangian multiplier αₚ and a kernel function K to calculate the the minimum classification hyperplane satisfying the quadratic programming model, which is prediction result ŷ′ᵢ of the target element content, and the calculation formula for predicting the target element content of any i^{th} sample to be measured being:
$${\hat{y}}_{i 1}^{′} = {\sum }_{p = 1}^{k} {α}_{p} {h}_{p} K + b ;$$
$${\hat{Y}}_{n 1}^{′} = {[ \begin{matrix}{\hat{y}}_{11}^{′} \\ {\hat{y}}_{21}^{′} \\ ⋮ \\ {\hat{y}}_{n 1}^{′}\end{matrix} ]}_{n \times 1} ,$$

7. The method of XRF quantitative analysis of heavy metal elements based on LLE-SVR according to claim 2, the fifth step of denormalizing the normalized output matrix of the target element content to obtain the denormalized output matrix, and calculating the coefficient of determination to evaluate the predicting effect of the LLE-SVR model comprises:
(1) denormalizing the normalized output matrix Ŷ′ₙ₁ in of the target element content to obtain the denormalized output matrix in Ŷ′ₙ₁:
$${\hat{y}}_{i 1}^{′} = {\hat{y}}_{i}^{′} ( {\hat{y}}_{max}^{′} - {\hat{y}}_{min}^{′} ) + {\hat{y}}_{min}^{′} ;$$
$${\hat{Y}}_{n 1} = {[ \begin{matrix}{\hat{y}}_{11} \\ {\hat{y}}_{21} \\ ⋮ \\ {\hat{y}}_{n 1}\end{matrix} ]}_{n \times 1} ;$$
wherein ŷᵢ₁ represents the prediction value of the target element content of the i^{th} sample of the denormalized output matrix and Ŷₙ₁, and y′ₘᵢₙ represent the minimum of the output matrix Ŷ′ₙ₁ , and y′ₘₐₓ represents the maximum of the output matrix Ŷ′ₙ₁; i=1, 2, . . . , n; and
(2) comparing the predicted target element content ŷᵢ₁ with the true target element content yᵢ₁, and calculating the coefficient of determination R²:
$${R}^{2} = 1 - \frac{{\sum }_{i = 1}^{n} {( {y}_{i 1} - {\hat{y}}_{i 1} )}^{2}}{{\sum }_{i = 1}^{n} {( {y}_{i 1} - {\overline{y}}_{i 1} )}^{2}} ;$$
$${\overline{y}}_{i 1} = \frac{{\sum }_{i = 1}^{n} {y}_{i 1}}{n} ;$$
wherein yᵢ₁represents the true value of the target element content of the i^{th} sample, and ŷᵢ₁ represents the prediction value of the target element content of the i^{th} sample, and y̅ᵢ₁ represents the average of the true value of the target element content of the i^{th} sample to be measured; i=1, 2, . . . , n.

8. A computer device, comprising a storage storing a computer program, and a processor configured to perform the steps of the method of XRF quantitative analysis of heavy metal elements based on LLE-SVR as claimed in claim 1 when executing the computer program.

9. A computer-readable storage medium storing a computer program, wherein a processor is configured to perform the steps of the method of XRF quantitative analysis of heavy metal elements based on LLE-SVR as claimed in claim 1 when executing the computer program.

10. An information data processing terminal, configured to implement the method of XRF quantitative analysis of heavy metal elements based on LLE-SVR as claimed claim 1.
